(19) United States
(12) Patent Application Publication
(10) Pub. No.: US 2025/0037875 A1
(43) Pub. Date: Jan. 30, 2025

(54) CALCULATION METHOD

(71) Applicant: NEC Corporation, Minato-ku, Tokyo (JP)

(72) Inventors: Tasuku Kitade, Tokyo (JP); Masanori Tsujikawa, Tokyo (JP)

(73) Assignee: NEC Corporation, Minato-ku, Tokyo (JP)

(21) Appl. No.: 18/716,193

(22) PCT Filed: Dec. 21, 2021
(86) PCT No.: PCT/JP2021/047378

§ 371 Date: Jun. 4, 2024

(51) Int. Cl. G16H 50/30; A61B 5/16

(52) U.S. Cl. CPC G16H 50/30; A61B 5/165

(57) ABSTRACT

A stress value calculating device 100 of the present invention includes: a minimum feature value calculating unit 121 that acquires biometric data obtained by measurement in a time series from a person, and calculates, as a minimum feature value, a feature value of each preset minimum time unit of the acquired biometric data; a first feature value calculating unit 122 that calculates, after passage of a first time unit which is a time unit longer than the minimum time unit, as a first feature value, a feature value of biometric data obtained by measurement in the first time unit using the minimum feature value corresponding to the biometric data of the first time unit; and a calculating unit 123 that calculates a value representing the physical condition of the person using information based on the first feature value.

### TECHNICAL FIELD

The present invention relates to a calculation method, a calculating device, and a program for calculating a value representing the physical condition of a person.

### BACKGROUND ART

As methods of calculating a stress value of a person, there are known methods using biometric data such as a heart rate of the person. In particular, in recent years, more people wear wearable terminals such as smart watches, accordingly it is easy to acquire biometric data from people always and for a mid- to long-term, and chronic stress values are calculated using the mid- to long-term biometric data. For example, mid- to long-term biometric data of several hours, several days, one month, or the like is acquired, and chronic stress values are calculated in some cases. In addition, as an example, Patent Literature 1 describes acquisition of biometric data from a wearable terminal attached to a person, and estimation of chronic stress.

### CITATION LIST

### Patent Literature

Patent Literature 1: JP 2010-184041 A

### SUMMARY OF INVENTION

### Technical Problem

However, in a case where a stress value is calculated using mid- to long-term biometric data as mentioned above, it is necessary to process together a large amount of entire biometric data of a mid- to long-term. Then, there is a problem that a process of calculation of the stress value takes a long time, and it is difficult to promptly calculate the stress value. In addition, what is difficult to calculate is not limited to stress, but it is similarly difficult to promptly calculate a value representing the physical condition of a person like physical and mental fatigue, or inner condition.

Therefore, an object of the present invention is to provide a calculation method that can solve the problem mentioned above that a process of calculation of a value representing physical condition takes a long time, and it is difficult to promptly calculate the value representing the physical condition.

### Solution to Problem

A person calculation method according to one aspect of the present invention includes: acquiring biometric data obtained by measurement in a time series from a person, and calculating, as a minimum feature value, a feature value of each preset minimum time unit of the acquired biometric data:
after passage of a first time unit which is a time unit longer than the minimum time unit, calculating, as a first feature value, a feature value of biometric data obtained by measurement in the first time unit using the minimum feature value corresponding to the biometric data of the first time unit; and
calculating a value representing physical condition of the person using information based on the first feature value.

In addition, a calculating device according to one aspect of the present invention includes:
a minimum feature value calculating unit that acquires biometric data obtained by measurement in a time series from a person, and calculates, as a minimum feature value, a feature value of each preset minimum time unit of the acquired biometric data;
a first feature value calculating unit that calculates, after passage of a first time unit which is a time unit longer than the minimum time unit, as a first feature value, a feature value of biometric data obtained by measurement in the first time unit using the minimum feature value corresponding to the biometric data of the first time unit; and
a calculating unit that calculates a value representing physical condition of the person using information based on the first feature value.

In addition, a program according to one aspect of the present invention causes an information processing device to execute processes of:
acquiring biometric data obtained by measurement in a time series from a person, and calculating, as a minimum feature value, a feature value of each preset minimum time unit of the acquired biometric data;
after passage of a first time unit which is a time unit longer than the minimum time unit, calculating, as a first feature value, a feature value of biometric data obtained by measurement in the first time unit using the minimum feature value corresponding to the biometric data of the first time unit; and
calculating a value representing physical condition of the person using information based on the first feature value.

### Advantageous Effects of Invention

By being configured in the manners described above, the present invention makes it possible to calculate a value representing the physical condition promptly.

### BRIEF DESCRIPTION OF DRAWINGS

FIG. 1 is a block diagram depicting the configuration of a stress value calculating device in a first exemplary embodiment of the present invention.

FIG. 2 is a figure depicting a state of data processing by the stress value calculating device disclosed in FIG. 1.

FIG. 3 is a figure depicting a state of data processing by the stress value calculating device disclosed in FIG. 1.

FIG. 4 is a figure depicting a state of data processing by the stress value calculating device disclosed in FIG. 1.

FIG. 5 is a flowchart depicting an operation performed by the stress value calculating device disclosed in FIG. 1.

FIG. 6 is a flowchart depicting an operation performed by the stress value calculating device disclosed in FIG. 1.

FIG. 7 is a flowchart depicting an operation performed by the stress value calculating device disclosed in FIG. 1.

FIG. 8 is a block diagram depicting the hardware configuration of a calculating device in a second exemplary embodiment of the present invention.

FIG. 9 is a block diagram depicting the configuration of the calculating device in the second exemplary embodiment of the present invention.

FIG. 10 is a flowchart depicting an operation performed by the calculating device in the second exemplary embodiment of the present invention.

### DESCRIPTION OF EMBODIMENTS

### First Exemplary Embodiment

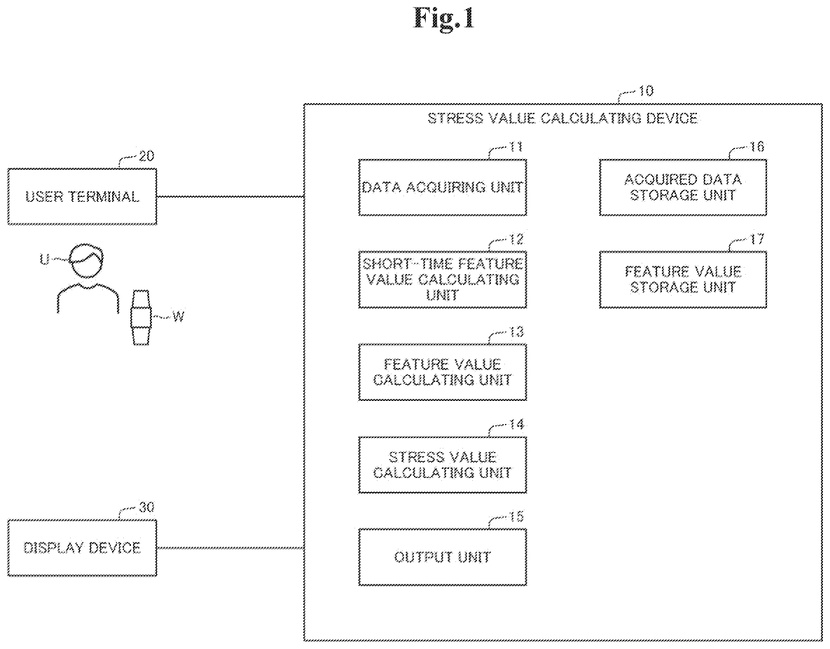

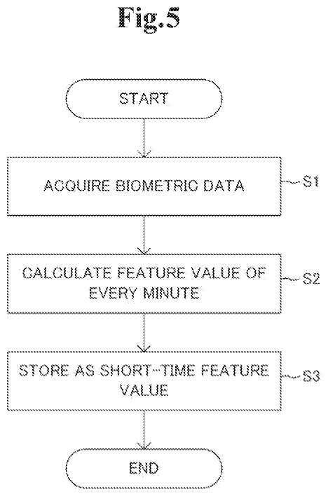

A first exemplary embodiment of the present invention is explained with reference to FIG. 1 to FIG. 7. FIG. 1 to FIG. 4 are figures for explaining the configuration of a stress value calculating device, and FIG. 5 to FIG. 7 are figures for explaining processing operations performed by the stress value calculating device.

### Configuration

A stress value calculating device 10 (calculating device) in the present invention is used for calculating a stress value representing a stress-related state of a person. For example, the stress value calculating device 10 is used for calculating a value representing chronic stress chronically put on a person. It should be noted that the stress value calculating device 10 in the present invention may calculate any stress value of a person. In addition, the present invention can be applied not only to calculation of the stress value, but also to calculation of a value representing the physical condition of a person like physical and mental fatigue, or inner condition. That is, a stress value mentioned in the present embodiment is an example of a value representing the physical condition of an estimation-target person, and, as other examples of the value representing the physical condition, the value may be any value such as a fatigue degree representing the degree of fatigue, or some index value representing condition.

The stress value calculating device 10 is configured using one or more information processing devices including an arithmetic device and a storage device. Then, as depicted in FIG. 1, the stress value calculating device 10 includes a data acquiring unit 11, a short-time feature value calculating unit 12, a feature value calculating unit 13, a stress value calculating unit 14, and an output unit 15. Respective functions of the data acquiring unit 11, the short-time feature value calculating unit 12, the feature value calculating unit 13, the stress value calculating unit 14, and the output unit 15 can be realized by the arithmetic device executing programs that are stored on the storage device, and are for realizing the respective functions. In addition, the stress value calculating device 10 includes an acquired data storage unit 16 and a feature value storage unit 17. The acquired data storage unit 16 and the feature value storage unit 17 are configured using the storage device. Hereinbelow; the respective configurations are mentioned in detail.

The data acquiring unit 11 acquires data to be used for calculating a stress value of a person. Specifically; the data acquiring unit 11 acquires biometric data about a person U when the person U is leading an everyday life, when the person U is doing her/his duties at the workplace or the like, and so on. For example, the biometric data is various information generated from the body of the person, and, as an example, is a heart rate, an acceleration, a sweat rate, and the like. Such biometric data is always obtained by measurement in a time series by a measurement device such as a wearable terminal W wom by the person U as depicted in FIG. 1, and is uploaded from the measurement device to the stress value calculating device 10 via a user terminal 20 such as a smartphone operated by the user.

Here, timings at which the biometric data is uploaded from the wearable terminal W and the user terminal 20 to the stress value calculating device 10 become irregular depending on the processing status, communication status, and the like of each terminal or device, in some cases. In addition, the duration over which the wearable terminal W and the user terminal 20 upload the biometric data to the stress value calculating device 10 also is not constant depending on the processing status, the communication status, and the like, in some cases. Therefore, the data acquiring unit 11 acquires irregular amounts of the biometric data irregularly from the wearable terminal W and the user terminal 20, and the biometric data can be often acquired with a delay from the time of measurement for the biometric data. As an example, there can be a delay of one hour from the time of last measurement for the biometric data of two hours during which measurement has been performed on the person until the time of acquisition of the biometric data by the data acquiring unit 11.

Then, the data acquiring unit 11 temporarily stores the acquired biometric data on the acquired data storage unit 16 in association the person and measurement time. It should be noted that the data acquiring unit 11 may acquire the biometric data obtained by measurement using any measurement device. Note that the acquired data storage unit 16 may not be provided, and the data acquiring unit 11 may send the acquired biometric data to the short-time feature value calculating unit 12 without storing it.

When the data acquiring unit 11 acquires the biometric data as mentioned above, the short-time feature value calculating unit 12 (minimum feature value calculating unit) calculates feature values of the biometric data. Specifically, the short-time feature value calculating unit 12 divides the biometric data of predetermined duration into minimum time units that are preset in a time series, calculates a feature value from biometric data of each divided minimum time unit, and stores, as short-time feature values (minimum feature values) and on the feature value storage unit 17, the feature values in association with time during which the source biometric data has been obtained by measurement.

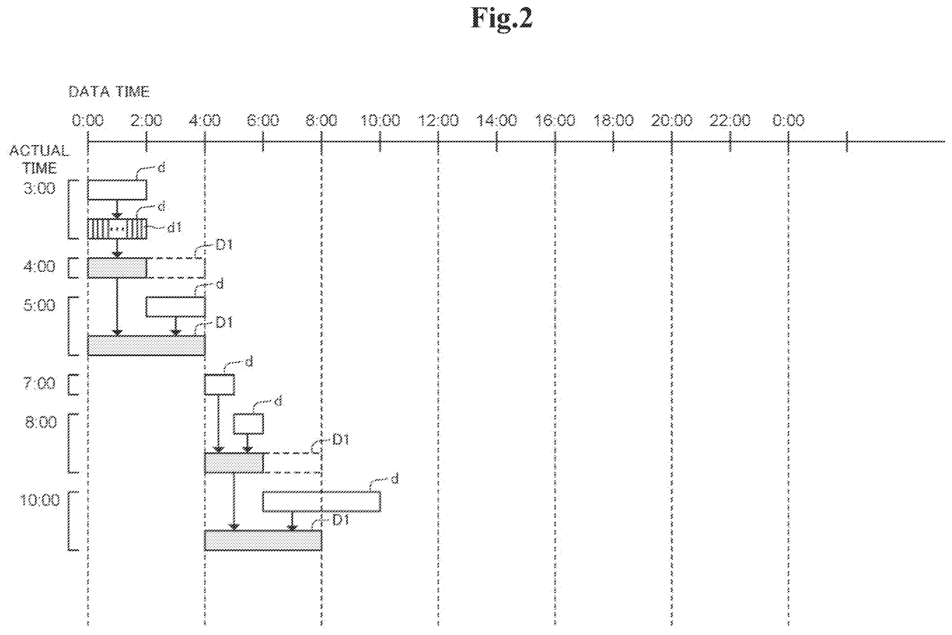

Here, a process performed by the short-time feature value calculating unit 12 is explained with reference to FIG. 2. It is assumed that, in FIG. 2, the horizontal axis represents time of measurement for biometric data, and the vertical axis represents actual times. Note that it is assumed in FIG. 2 that measurement for the biometric data is started from the measurement time “0:00.”

First, in the following case to be explained, it is assumed that the data acquiring unit 11 acquires biometric data d of two hours in the measurement time “0:00-2:00” from the actual time “3:00” in FIG. 2. In this case, the short-time feature value calculating unit 12 divides the acquired biometric data d into data of “one minute” which is set as a minimum time unit, calculates a feature value of each piece of biometric data of “one minute,” and stores, as short-time feature values d1, the feature values in association with times at intervals of one minute starting from “0:00.” At this time, as a feature value, for example, the average, variance/standard deviation, maximum value, minimum value, quartile, or the like of the biometric data is calculated. For simplicity of explanation, it is assumed in the present embodiment that the average of the biometric data is calculated as a feature value.

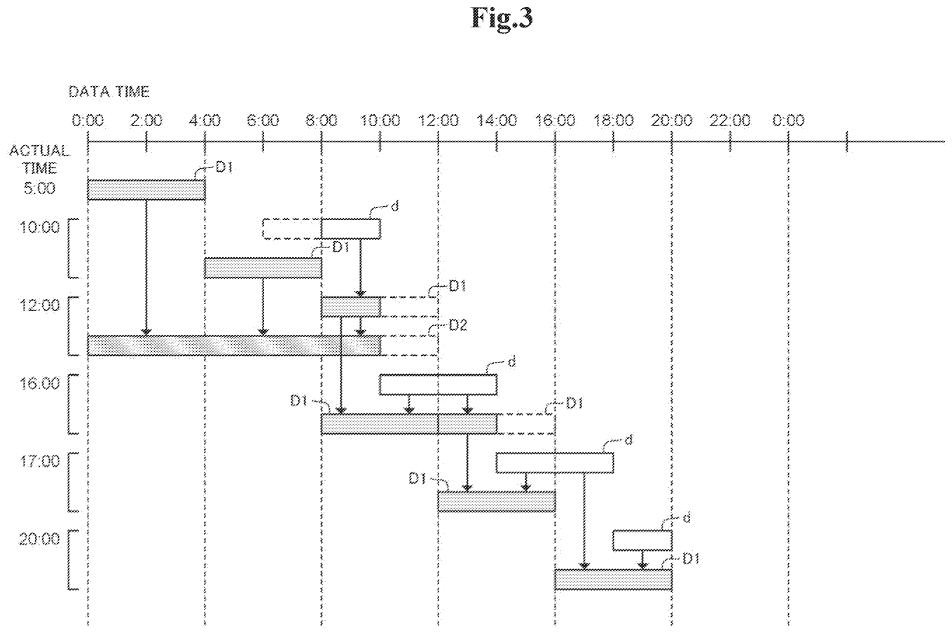

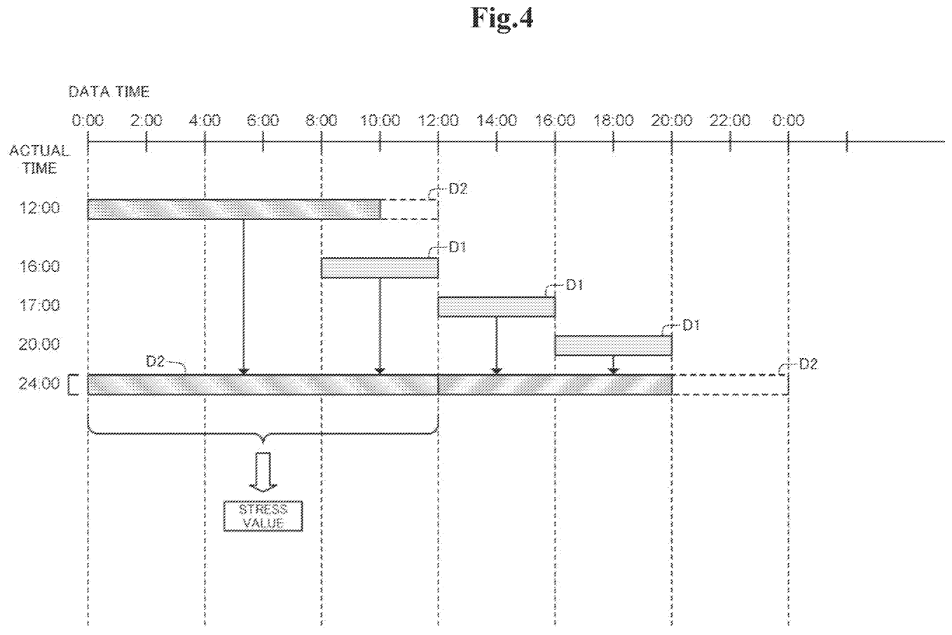

Then, calculation of short-time feature values d1 by the short-time feature value calculating unit 12 is executed immediately upon acquisition of biometric data d by the data acquiring unit 11. Therefore, upon acquisition of biometric data d of two hours of the measurement time “2:00-4:00” at the actual time “5:00” depicted in FIG. 2, the short-time feature value calculating unit 12 immediately calculates and stores a short-time feature value of each minimum time unit. Note that although calculation of short-time feature values d1 for biometric data d acquired at the actual time “5:00” is not depicted, but omitted in the example depicted in FIG. 2, short-time feature values are calculated similarly to the manner mentioned above. In addition, although calculation of short-time feature values d1 from biometric data d is similarly not depicted, but omitted regarding other times in FIG. 2 and in FIGS. 3 and 4, short-time feature values are calculated similarly to the manner mentioned above.

The feature value calculating unit 13 (first feature value calculating unit) has a function of calculating, first, a 4-hour feature value (first feature value) as a feature value of biometric data of “four hours” set as a first time unit, using short-time feature values calculated from biometric data as mentioned above. Here, it is assumed in the present embodiment that the first time unit is set to “four hours” longer than “one minute,” which is an example of the minimum time unit mentioned above. It should be noted that the first time unit may be set to any time as long as it is time longer than the minimum time unit.

Upon passage of “four hours,” which is the first time unit as a target period of biometric data, the feature value calculating unit 13 calculates a 4-hour feature value using short-time feature values corresponding to biometric data obtained by measurement in the four hours, and stores the 4-hour feature value on the feature value storage unit 17. At this time, first, upon the passage of the four hours as the target period, even in a case where biometric data of the entire four hours has not been acquired, the feature value calculating unit 13 calculates the 4-hour feature value using only short-time feature values corresponding to biometric data having already been acquired, and stores the 4-hour feature value on the feature value storage unit 17 in advance. Thereafter, the feature value calculating unit 13 checks whether or not biometric data of the four hours as the target period has been newly acquired, and, in a case where biometric data has been newly acquired, the feature value calculating unit 13 newly calculates the 4-hour feature value using short-time feature values corresponding to the new biometric data, and the 4-hour feature value having already been calculated and stored, and updates and stores the 4-hour feature value. Note that in a case where biometric data of the entire four hours has not been acquired, every time one hour, which is a time interval shorter than the first time unit, passes, the feature value calculating unit 13 checks whether or not biometric data of the four hours as the target period has been newly acquired. It should be noted that time intervals at which it is checked whether or not biometric data of the four hours as the target period has been newly acquired are not limited to one hour, but may be any time interval.

Then, after biometric data of the entire four hours as the target period has been acquired, and the feature value calculating unit 13 calculates the 4-hour feature value using short-time feature values corresponding to the biometric data, and stores the 4-hour feature value, the feature value calculating unit 13 changes the target period to the subsequent four hours. Then, after another four hours have passed, a process similar to the process mentioned above is performed, the subsequent 4-hour feature value is calculated using short-time feature values corresponding to biometric data obtained by measurement in the subsequent four hours, and stored on the feature value storage unit 17.

Note that, even in a case where biometric data of the entire four hours as the target period has not been actually acquired, the data feature value calculating unit 13 treats biometric data as if biometric data of the entire time has been acquired, in some cases. This is because there is a possibility that there is undesirably a time period during which biometric data cannot be acquired for some reason. Therefore, for example, in a case where, after four hours as the target period have passed, biometric data of time after the four hours has been acquired, in a case where preset time has passed after the four hours as the target period have passed, or in other cases, the data feature value calculating unit 13 treats biometric data as if biometric data of the entire four hours as the target period has been acquired even in a case where there is a period during which biometric data has not been acquired in the target period. Then, a 4-hour feature value is calculated using short-time feature values of only biometric data having been acquired, and the target period is changed to the subsequent four hours.

Here, a process of calculation of a 4-hour feature value performed by the feature value calculating unit 13 is explained with reference to FIG. 2. First, assuming that the target period is four hours of the measurement time “0:00-4:00,” the feature value calculating unit 13 calculates a 4-hour feature value at the actual time “4:00.” At this time, in the example depicted in FIG. 2, since only biometric data d of two hours of the measurement time “0:00-2:00” has been acquired before the actual time “4:00,” a 4-hour feature value D1 of the measurement time “0:00-4:00” is calculated from only short-time feature values d1 corresponding to the biometric data d of the time “0:00-2:00,” and is stored in advance. Thereafter, every time one hour passes, the feature value calculating unit 13 checks whether or not biometric data of the four hours as the target period has been newly acquired. Then, in the example depicted in FIG. 2, since one hour has passed at the actual time “5:00,” the feature value calculating unit 13 checks whether or not biometric data of the four hours as the target period has been newly acquired. Then, biometric data d of two hours of the measurement time “2:00-4:00” is acquired at the actual time “5:00.” and short-time feature values, which are not depicted, are calculated and stored. Therefore, the feature value calculating unit 13 updates the 4-hour feature value D1 using the short-time feature values corresponding to the biometric data d of the measurement time “2:00-4:00” acquired at the actual time “5:00,” and the 4-hour feature value D1 corresponding to the biometric data d of “0:00-2:00” having already been calculated and stored. Thereby, the 4-hour feature value D1 based on the biometric data of the entire measurement time “0:00-4:00” as the target period is calculated and stored at the time point of the actual time “5:00.” It should be noted that the feature value calculating unit 13 may calculate the 4-hour feature value D1 using all the short-time feature values having been acquired, in this case the short-time feature values corresponding to the biometric data d of the measurement time “0:00-2:00,” and the short-time feature values corresponding to the biometric data d of the measurement time “2:00-4:00.”

Note that in a case where short-time feature values are the averages of biometric data as mentioned above, it is sufficient if a 4-hour feature value is simply determined as the average of the short-time feature values, and, in a case where there is a 4-hour feature value having already been calculated, it is sufficient if the 4-hour feature value is determined as the average taking into consideration time of the 4-hour feature value and time of new short-time feature values. Therefore, the feature value calculating unit 13 can calculate the 4-hour feature value faster than in a case where a 4-hour feature value is calculated from biometric data itself.

Thereafter, the feature value calculating unit 13 changes the target period to the measurement time “4:00-8:00,” which is the subsequent four hours. Therefore, the feature value calculating unit 13 performs a 4-hour feature value calculation process similarly to the manner mentioned above when the actual time “8:00” has come after passage of the next four hours.

In addition, the feature value calculating unit 13 (second feature value calculating unit) has a function of calculating a 12-hour feature value (second feature value) as a feature value of biometric data of “12 hours” set as a second time unit, using 4-hour feature values (first feature values) calculated as mentioned above. Here, it is assumed in the present embodiment that the second time unit is set to “12 hours” longer than “four hours,” which is an example of the first time unit mentioned above. It should be noted that the second time unit may be set to any time as long as it is time longer than the first time unit.

Upon passage of “12 hours,” which is the second time unit as a target period of biometric data, the feature value calculating unit 13 calculates a 12-hour feature value using 4-hour feature values corresponding to biometric data obtained by measurement in the 12 hours, and stores the 12-hour feature value on the feature value storage unit 17. At this time, first, upon the passage of the 12 hours as the target period, even in a case where biometric data of the entire 12 hours has not been acquired, the feature value calculating unit 13 calculates the 12-hour feature value using only 4-hour feature values corresponding to biometric data having already been acquired, and stores the 12-hour feature value on the feature value storage unit 17 in advance. Thereafter, every time 12 hours as the target period have passed, the feature value calculating unit 13 newly calculates the 12-hour feature value using 4-hour feature values having been newly calculated and stored, and the 12-hour feature value having already been calculated and stored, and updates and stores the 12-hour feature value.

Here, a process of calculation of a 12-hour feature value performed by the feature value calculating unit 13 is explained with reference to FIG. 3 to FIG. 4. Note that, in FIG. 3, part of data processing depicted in FIG. 2 is not depicted, but omitted, and processes of the still subsequent actual times are added, and, in FIG. 4, part of data processing depicted in FIG. 3 is not depicted, but omitted, and processes of the still subsequent actual times are added.

First, assuming that the target period is 12 hours of the measurement time “0:00-12:00.” the feature value calculating unit 13 calculates a 12-hour feature value at the actual time “12:00.” At this time, in the example depicted in FIG. 3, since only 4-hour feature values D1 corresponding to biometric data d of 10 hours of the measurement time “0:00-10:00” has been generated before the actual time “12:00,” a 12-hour feature value D2 of the measurement time “0:00-12:00” is calculated from only the 4-hour feature values D1 corresponding to the biometric data d of the time “0:00-10:00,” and is stored in advance.

Thereafter, the feature value calculating unit 13 waits for calculation of the 12-hour feature value D2 until the subsequent 12 hours pass, and when the actual time “0:00,” at which the next 12 hours have passed, has come, calculates the 12-hour feature value D2. At this time, since the 12-hour feature value D2 corresponding to the 12 hours of the measurement time “0:00-12:00” does not reflect entire biometric data d, the feature value calculating unit 13 calculates also a 12-hour feature value D2 corresponding to the 12 hours of the measurement time “0:00-12:00,” and calculates a 12-hour feature value D2 corresponding to the still next 12 hours of the measurement time “12:00-0:00.” Then, in the example depicted in FIG. 4, since 4-hour feature values D1 corresponding to biometric data d of the entire time have been calculated for the 12 hours of the measurement time “0:00-12:00,” the feature value calculating unit 13 newly calculates the 12-hour feature value D2 of the measurement time “0:00-12:00” using newly calculated 4-hour feature values D1 corresponding to biometric data d of the measurement time “8:00-12:00,” and the 12-hour feature value D2 of the measurement time “0:00-10:00” having already been calculated, and updates and stores the 12-hour feature value D2 in advance. At this time, when the 12-hour feature value D2 of the measurement time “0:00-12:00” is newly calculated, since data of the measurement time “8:00-10:00” overlaps, the 12-hour feature value D2 needs to be calculated taking into consideration the overlapping data. In addition, regarding the still next 12 hours of the measurement time “12:00-0:00,” a 12-hour feature value D2 is calculated from only 4-hour feature values D1 corresponding to biometric data d of the time “12:00-20:00” having been calculated, and is stored in advance. It should be noted that the feature value calculating unit 13 may calculate the 12-hour feature value D2 using 4-hour feature values D1 of the entire target period, in this case 4-hour feature values D1 corresponding to biometric data d of the measurement time “0:00-4:00,” 4-hour feature values D1 corresponding to biometric data d of the measurement time “4:00-8:00,” and 4-hour feature values D1 corresponding to biometric data d of the measurement time “8:00-12:00.”

Note that in a case where 4-hour feature values are the averages of biometric data as mentioned above, it is sufficient if a 12-hour feature value is simply determined as the average of the 4-hour feature values, and, in a case where there is a 12-hour feature value having already been calculated, it is sufficient if the 12-hour feature value is determined as the average taking into consideration time of the 12-hour feature value and time of new 4-hour feature values. Therefore, the feature value calculating unit 13 can calculate the 12-hour feature value faster than in a case where a 12-hour feature value is calculated from biometric data of 12 hours itself.

It should be noted that the feature value calculating unit 13 does not necessarily calculate a 12-hour feature value D2 every time 12 hours have passed, but may calculate a 12-hour feature value D2 every time preset time shorter than 12 hours has passed. For example, the feature value calculating unit 13 may check whether or not 4-hour feature values have been newly calculated every hour, and may calculate a new: 12-hour feature value D2 every time new 4-hour feature values have been calculated.

When the feature value calculating unit 13 has calculated a 12-hour feature value D2 corresponding to biometric data of the entire target period as mentioned above, the stress value calculating unit 14 (calculating unit) calculates a stress value of the person using the 12-hour feature value D2. Therefore, in the example depicted in FIG. 4, since a 12-hour feature value D2 about 12 hours of the measurement time “0:00-12:00” of the previous day is calculated at the actual time “0:00,” a stress value is calculated using the 12-hour feature value D2. Note that the stress value calculating unit 14 may calculate the stress value by any method from the 12-hour feature value D2, and may calculate the stress value using other information.

Note that the stress value calculating unit 14 does not necessarily calculate a stress value from a 12-hour feature value D2 calculated as mentioned above. For example, the stress value calculating unit 14 may calculate a stress value using a 12-hour feature value D2 calculated for a period during which there is a period during which biometric data has not been acquired. In addition, at a preset time, the stress value calculating unit 14 may calculate a stress value from a 12-hour feature value D2 at the time point. In this case, the feature value calculating unit 13 mentioned above may calculate the 12-hour feature value D2 of the immediately preceding 12 hours from the time point from 4-hour feature values D1 having been calculated in the past. As an example, in a case depicted in FIG. 4, the feature value calculating unit 13 is configured to calculate stress values three times a day at intervals of eight hours, that is, at 4:00, 12:00, and 20:00. Then, at the time point of 12:00, a 12-hour feature value D2 corresponding to biometric 20) data of “0:00-10:00” (biometric data of 10:00-12:00 has not been acquired) is calculated, and a stress value is calculated from the 12-hour feature value D2. Then, at the time point of 20:00, a 12-hour feature value D2 corresponding to biometric data of “8:00-20:00” is calculated using 4-hour feature values D1 of “8:00-12:00.” “12:00-16:00,” and “16:00-20:00,” and a stress value is calculated from the 12-hour feature value D2.

The output unit 15 outputs information based on the stress value calculated at the stress value calculating unit 14 as mentioned above. For example, every time the stress value is calculated, in a case where the stress value exceeds a preset criterion value, on the basis of which it is determined whether stress is high, the output unit 15 outputs an instruction to cause a display 30) device 30 of an information processing device operated by an administrator at the workplace of the person U, a family member of the person U, or the like to display information to that effect (alert). Alternatively, every time the stress value is calculated, the output unit 15 may always output an instruction such that the stress value itself, that is, time-series changes of the stress value of the person U, is displayed, or may output any data based on the stress value. In addition, the output unit 15 may output data based on the stress value to any person such as the target person U.

### Operation

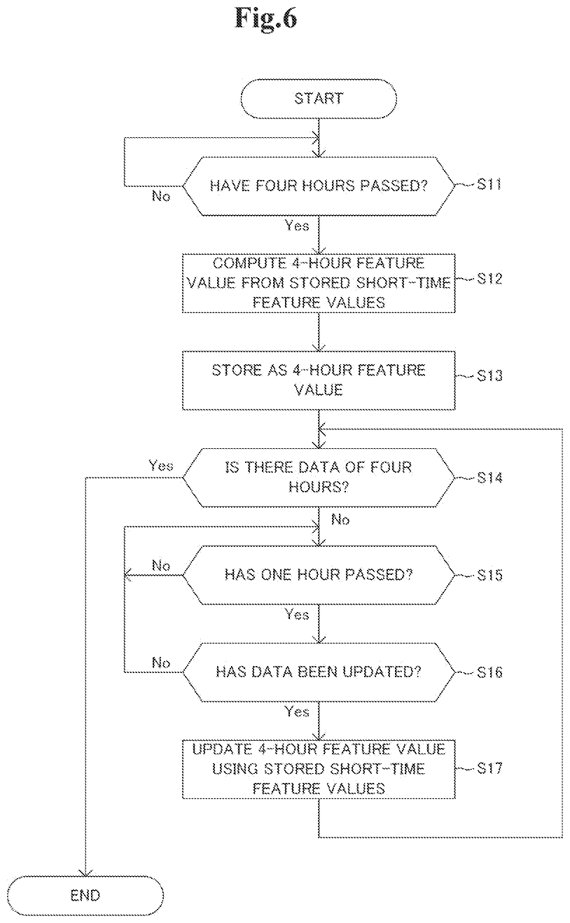

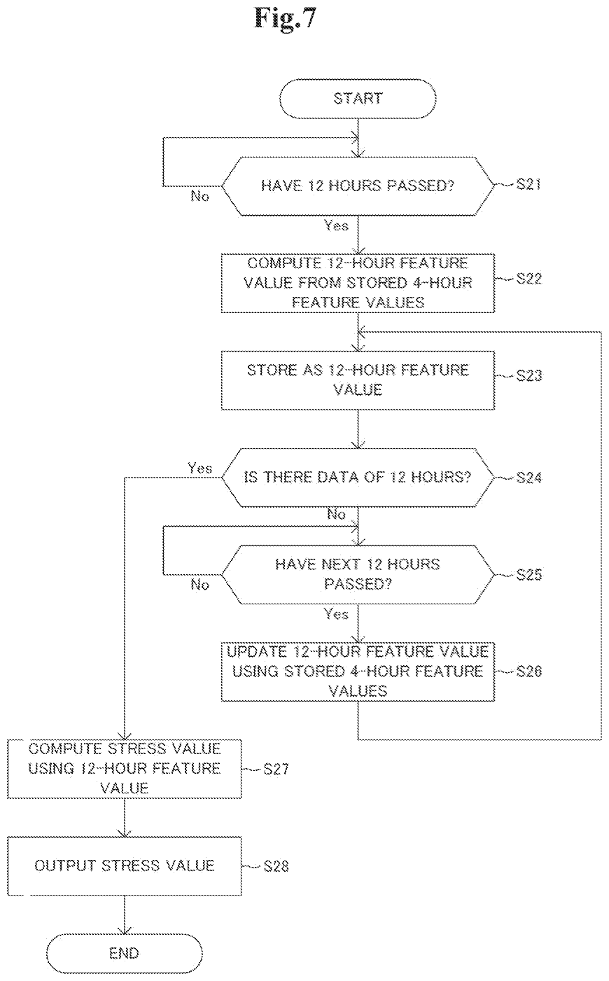

Next, operations performed by the stress value calculating device 10 mentioned above are explained mainly with reference to flowcharts in FIG. 5 to FIG. 7. FIG. 5 depicts operations performed by the data acquiring unit 11 and the short-time feature value calculating unit 12 of the stress value calculating device 10. FIG. 6 depicts an operation of calculation of a 4-hour feature value performed by the feature value calculating unit 13 of the stress value calculating device 10, and FIG. 7 depicts an operation of calculation of a 12-hour feature value performed by the feature value calculating unit 13. Note that, hereinbelow, a situation where biometric data is acquired as depicted in FIG. 2 to FIG. 4 is explained along actual times as an example. Note that it is assumed in this example that measurement for biometric data is started from the measurement time “0:00.”

First, as depicted in FIG. 2, at the actual time “3:00,” which is after the start of measurement for biometric data, the data acquiring unit 11 acquires biometric data d of two hours of the measurement time “0:00-2:00” (step S1 in FIG. 5). Then, the short-time feature value calculating unit 12 divides the acquired biometric data d into data of “one minute” which is set as the minimum time unit, and calculates a feature value of each piece of biometric data of “one minute” (step S2 in FIG. 5). Then, the short-time feature value calculating unit 12 stores the calculated feature values as short-time feature values d1 in association times from “0:00” to “2:00” at intervals of one minute (step S3 in FIG. 5).

Thereafter, when the actual time “4:00” in FIG. 2 has come, this means that the target period for which the feature value calculating unit 13 calculates a 4-hour feature value has passed, that is, four hours of the measurement time “0:00-4:00” have passed (Yes at step S11 in FIG. 6). Then, the feature value calculating unit 13 calculates a 4-hour feature value D1 from the short-time feature values d1 corresponding to the biometric data d included in the four hours of the measurement time “0:00-4:00,” and stores the 4-hour feature value D1 (steps S12 and S13 in FIG. 6). At this time, in the example depicted in FIG. 2, since only biometric data d of two hours of the measurement time “0:00-2:00” has been acquired, a 4-hour feature value D1 of the measurement time “0:00-4:00” is calculated from only short-time feature values d1 corresponding to the biometric data d of the time “0:00-2:00,” and is stored in advance.

Note that, since biometric data of the entire four hours of the measurement time “0:00-4:00” has not been acquired at this time point (No at step S14 in FIG. 6), it is checked every hour since then whether or not biometric data of the four hours as the target period has been newly acquired (Yes at step S15, and S16 in FIG. 6). Then, in a case where biometric data has been newly acquired (Yes at step S16 in FIG. 6), the feature value calculating unit 13 newly calculates the 4-hour feature value using short-time feature values d1 corresponding to the new biometric data, and the 4-hour feature value D1 having already been calculated and stored, and updates and stores the 4-hour feature value D1 (step S17 in FIG. 6). Note that in a case where biometric data of time after the four hours as the target period has been acquired, it is determined that biometric data of the entire four hours has been acquired even in a case where there is time during which biometric data has not been acquired, and the 4-hour feature value D1 is calculated.

Thereafter, when the actual time “5:00” in FIG. 2 has come, the data acquiring unit 11 acquires biometric data d of the two hours of the measurement time “2:00-4:00” (step S1 in FIG. 5). Then, the short-time feature value calculating unit 12 calculates a short-time feature value d1 of each “one minute” from the acquired biometric data d similarly to the manner mentioned above, and stores the short-time feature values d1 (steps S2 and S3 in FIG. 5). In addition, since, at the actual time “5:00” in FIG. 2, one hour has passed after the calculation of the previous 4-hour feature value D1 (Yes at step S15 in FIG. 6), it is checked whether or not biometric data d of the four hours as the target period has been newly acquired (step S16 in FIG. 6). At this time, since the data acquiring unit 11 has acquired new biometric data d of the two hours of the measurement time “2:00-4:00” (Yes at step S16 in FIG. 6), the feature value calculating unit 13 calculates a new: 4-hour feature value D1 using short-time feature values d1 corresponding to the new biometric data d, and the 4-hour feature value D1 having already been calculated and stored, and updates and stores the new 4-hour feature value D1 (step S17 in FIG. 6). Thereby, since biometric data of the entire four hours of the measurement time “0:00-4:00” has been acquired (Yes at step S14 in FIG. 6), calculation of the 4-hour feature value D1 corresponding to the measurement time “0:00-4:00” ends.

Thereafter, similarly to the manner mentioned above, calculation of short-time feature values d1 every time biometric data d is acquired, and calculation of a 4-hour feature value D1 of the measurement time “4:00-8:00” are performed. Specifically; as depicted in FIG. 2, the data acquiring unit 11 acquires biometric data d at each of the actual times “7:00” and “8:00,” and the short-time feature value calculating unit 12 calculates short-time feature values d1 of the acquired biometric data d. Then, since four hours of the measurement time “4:00-8:00” have passed at the actual time “8:00,” the feature value calculating unit 13 calculates a 4-hour feature value D1 from short-time feature values d1 corresponding to biometric data d included in the four hours of the measurement time “4:00-8:00,” and stores the 4-hour feature value D1. At this time, in the example depicted in FIG. 2, since only biometric data d of two hours of the measurement time “4:00-6:00” has been acquired, the feature value calculating unit 13 calculates a 4-hour feature value D1 of the measurement time “4:00-8:00” from only short-time feature values d1 corresponding to the biometric data d of the two hours, and stores the 4-hour feature value D1 in advance. Thereafter, the feature value calculating unit 13 checks whether new biometric data d has been acquired every hour, and, since biometric data d of the measurement time “6:00-10:00” is acquired when the actual time “10:00” has come, this means that biometric data d of the entire four hours of the measurement time “4:00-8:00” has been acquired. Therefore, the feature value calculating unit 13 newly calculates the 4-hour feature value D1 of the measurement time “4:00-8:00” using short-time feature values d1 corresponding to new biometric data d of two hours of the measurement time “6:00-8:00,” and the 4-hour feature value D1 having already been calculated and stored, and updates the 4-hour feature value D1.

Thereafter, since the next four hours of the measurement time “8:00-12:00” have passed when the actual time “12:00” depicted in FIG. 3 has come, a 4-hour feature value D1 is calculated similarly to the manner mentioned above. At this time, since there is only biometric data d of two hours of the measurement time “8:00-10:00,” the feature value calculating unit 13 calculates a 4-hour feature value D1 of the measurement time “8:00-12:00” from only short-time feature values d1 corresponding to the biometric data d of the two hours, and stores the 4-hour feature value D1 in advance.

Simultaneously, since 12 hours of the measurement time “0:00-12:00” have passed when the actual time “12:00” depicted in FIG. 3 has come (step S21 in FIG. 7), the feature value calculating unit 13 calculates a 12-hour feature value. Then, the feature value calculating unit 13 calculates a 12-hour feature value D2 using the 4-hour feature value D1 of the measurement time “0:00-4:00,” the 4-hour feature value D1 of the measurement time “4:00-8:00,” and the 4-hour feature value D1 of the measurement time “8:00-12:00,” and stores the 12-hour feature value D2 (steps S22 and S23 in FIG. 7). At this time, in the example depicted in FIG. 3, since only the biometric data d of 10 hours of the measurement time “0:00-10:00” has been acquired (No at step S24 in FIG. 7), calculation of a 12-hour feature value D2 is waited until the still next 12 hours passes (step S25 in FIG. 7). Note that, as depicted in FIG. 3, after the actual time “12:00” also, acquisition of new biometric data d and calculation of short-time feature values, and calculation of a 4-hour feature value are performed.

Thereafter, when the actual time “0:00,” at which the next 12 hours have passed, has come as depicted in FIG. 4 (Yes at step S25 in FIG. 7), the feature value calculating unit 13 calculates a 12-hour feature value (step S26 in FIG. 7). At this time, since biometric data d of the entire time has been acquired for the 12 hours of the measurement time “0:00-12:00,” and 4-hour feature values D1 of the entire time have been calculated, the feature value calculating unit 13 newly calculates the 12-hour feature value D2 of the measurement time “0:00-12:00” using newly calculated 4-hour feature values D1 corresponding to biometric data d of the measurement time “8:00-12:00,” and the 12-hour feature value D2 of the measurement time “0:00-10:00” having already been calculated, and updates and stores the 12-hour feature value D2 in advance. Note that, regarding the 12 hours of the measurement time “12:00-0:00,” a 12-hour feature value D2 is calculated from only 4-hour feature values D1 corresponding to biometric data d of the measurement time “12:00-20:00” having been calculated, and is stored in advance.

Then, when a 12-hour feature value D2 corresponding to biometric data of entire 12 hours of the measurement time “0:00-12:00” has been calculated as mentioned above, the stress value calculating device 10 calculates a stress value of the person using the 12-hour feature value D2 (step S27 in FIG. 7). Then, the stress value calculating device 10 outputs information based on the calculated stress value (step S28 in FIG. 7).

As mentioned above, in the present embodiment, short-time feature values are calculated every time biometric data is acquired, a 4-hour feature value is calculated using the short-time feature values, and furthermore a 12-hour feature value is calculated using the 4-hour feature values. Therefore, a 12-hour feature value can be calculated extremely faster than in a case where a 12-hour feature value is calculated from biometric data of 12 hours itself, and furthermore, by calculating a stress value from the feature value, the stress value can be calculated promptly.

### Second Exemplary Embodiment

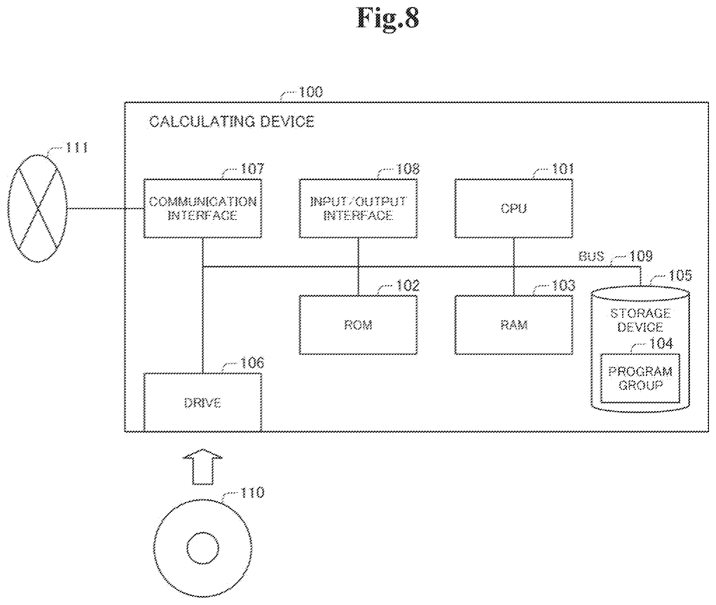

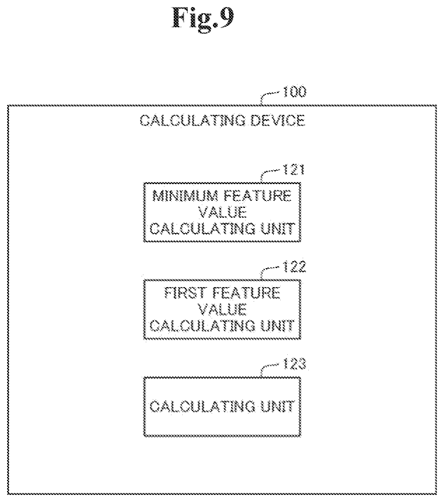

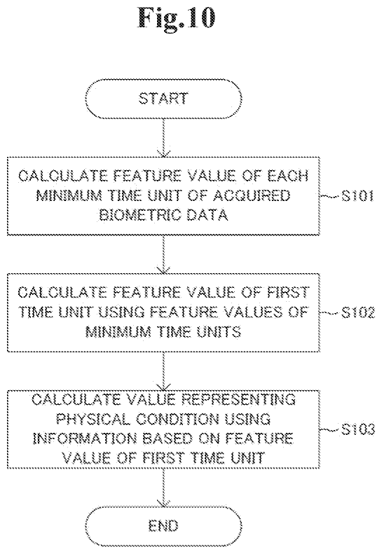

Next, a second exemplary embodiment of the present invention is explained with reference to FIG. 8 to FIG. 10. FIG. 8 to FIG. 9 are block diagrams depicting the configuration of a calculating device in the second exemplary embodiment, and FIG. 10 is a flowchart depicting an operation performed by the calculating device. Note that the present embodiment illustrates outlines of the configurations of the stress value calculating device and the stress value calculation method explained in the exemplary embodiment mentioned above.

First, the hardware configuration of a calculating device 100 in the present embodiment is explained with reference to FIG. 8. The calculating device 100 is configured using a typical information processing device, and has a hardware configuration as described below as an example.
Central Processing Unit (CPU) 101 (arithmetic device)
Read Only Memory (ROM) 102 (storage device)
Random Access Memory (RAM) 103 (storage device)
Program group 104 to be loaded to RAM 103
Storage device 105 having stored thereon the program group 104
Drive 106 that performs reading and writing on a storage medium 110 outside the information processing device
Communication interface 107 connected to a communication network 111 outside the information processing device
Input/output interface 108 for performing input/output of data
Bus 109 connecting the respective constituent elements

Then, the calculating device 100 can construct and have a minimum feature value calculating unit 121, a first feature value calculating unit 122, and a calculating unit 123 depicted in FIG. 9 through acquisition of the program group 104 and execution thereof by the CPU 101. Note that the program group 104 is stored on, for example, the storage device 105 or the ROM 102 in advance, is loaded to the RAM 103 by the CPU 101, and is executed by the CPU 101 as needed. In addition, the program group 104 may be supplied to the CPU 101 via the communication network 111, or may be stored on the storage medium 110 in advance, read out by the drive 106, and supplied to the CPU 101. It should be noted that the minimum feature value calculating unit 121, the first feature value calculating unit 122, and the calculating unit 123 mentioned above may be constructed using electronic circuits dedicated for realizing the means.

Note that FIG. 8 depicts an example of the hardware configuration of the information processing device that is the calculating device 100. The hardware configuration of the information processing device is not limited to that mentioned above. For example, the information processing device may be configured using part of the configuration mentioned above, such as without the drive 106.

Then, by functions of the minimum feature value calculating unit 121, the first feature value calculating unit 122, and the calculating unit 123 constructed by programs as mentioned above, the calculating device 100 executes the calculation method depicted in the flowchart in FIG. 10.

As depicted in FIG. 10, the calculating device 100 executes processes of:
acquiring biometric data obtained by measurement in a time series from a person, and calculating, as a minimum feature value, a feature value of each preset minimum time unit of the acquired biometric data (step S101);
after a first time unit which is a time unit longer than the minimum time unit has passed, calculating, as a first feature value, a feature value of the biometric data obtained by measurement in the first time unit using the minimum feature values corresponding to the biometric data of the first time unit (step S102); and
calculating a value representing the physical condition of the person using information based on the first feature value (step S103).

By being configured in the manners described above, the present invention calculates minimum feature values every time biometric data is acquired, calculates a first feature value using the minimum feature values, and calculates a value representing the physical condition of a person on the basis of the first feature value. Therefore, it is possible to calculate feature values extremely faster than in a case where feature values are calculated from biometric data of entire time itself, and it is possible to calculate a value representing the physical condition promptly.

Note that the programs mentioned above can be supplied to a computer by being stored on a non-transitory computer readable medium of any type. Non-transitory computer readable media include tangible storage media of various types. Examples of non-transitory computer readable media include a magnetic recording medium (e.g. flexible disc, magnetic tape, hard disk drive), a magneto-optical recording medium (e.g. magneto-optical disc), a CD-ROM (Read Only Memory), a CD-R, a CD-R/W, and a semiconductor memory (e.g. mask ROM, PROM (Programmable ROM), EPROM (Erasable PROM), flash ROM, and RAM (Random Access Memory)). In addition, the programs may also be supplied to a computer by being stored on a transitory computer readable medium of any type. Examples of transitory computer readable media include electric signals, optical signals, and electromagnetic waves. A transitory computer readable medium can supply programs to a computer via a wired communication channel such as an electric wire or an optical fiber, or a wireless communication channel.

While the present invention has been explained thus far with reference to the exemplary embodiments and the like described above, the present invention is not limited to the exemplary embodiments mentioned above. The configurations and details of the present invention can be changed within the scope of the present invention in various manners that can be understood by those skilled in the art. In addition, at least one or more functions of the functions of the minimum feature value calculating unit 121, the first feature value calculating unit 122, and the calculating unit 123 mentioned above may be executed at an information processing device installed and connected at any location on a network, that is, may be executed by so-called cloud computing.

### Supplementary Notes

Part of or the whole of the exemplary embodiments described above can also be described as in the following supplementary notes. Hereinbelow; outlines of the configurations of a calculation method, a calculating device, and a program in the present invention are explained. It should be noted that the present invention is not limited to the following configurations.

### (Supplementary Note 1)

A calculation method comprising:
acquiring biometric data obtained by measurement in a time series from a person, and calculating, as a minimum feature value, a feature value of each preset minimum time unit of the acquired biometric data;
after passage of a first time unit which is a time unit longer than the minimum time unit, calculating, as a first feature value, a feature value of biometric data obtained by measurement in the first time unit using the minimum feature value corresponding to the biometric data of the first time unit; and
calculating a value representing physical condition of the person using information based on the first feature value.

### (Supplementary Note 2)

The calculation method according to supplementary note 1, wherein, every time biometric data is acquired, a feature value of each minimum time unit of the acquired biometric data is calculated as the minimum feature value.

### (Supplementary Note 3)

The calculation method according to supplementary note 1 or 2, wherein, after the first feature value is calculated, a new first feature value is calculated using a new minimum feature value corresponding to newly acquired biometric data of the first time unit, and the first feature value having already been calculated.

### (Supplementary Note 4)

The calculation method according to supplementary note 3, wherein, in a case where the first feature value has not been calculated using the minimum feature value corresponding to biometric data of entire time in the first time unit, it is checked whether or not biometric data of the first time unit has been newly acquired, and, in a case where biometric data has been newly acquired, a new first feature value is calculated using a new minimum feature value corresponding to the newly acquired biometric data, and the first feature value having already been calculated.

### (Supplementary Note 5)

The calculation method according to supplementary note 4, wherein, in a case where the first feature value has not been calculated using the minimum feature value corresponding to biometric data of entire time in the first time unit, it is checked whether or not biometric data of the first time unit has been newly acquired at a time interval shorter than the first time unit, and, in a case where biometric data has been newly acquired, a new first feature value is calculated using a new minimum feature value corresponding to the newly acquired biometric data, and the first feature value having already been calculated.

### (Supplementary Note 6)

The calculation method according to any one of supplementary notes 1 to 5, wherein
the calculation method comprises, after passage of a second time unit which is a time unit longer than the first time unit, calculating, as a second feature value, a feature value of biometric data obtained by measurement in the second time unit using the first feature value corresponding to the biometric data included in the second time unit, and
a value representing physical condition is calculated on a basis of the second feature value.

### (Supplementary Note 7)

The calculation method according to supplementary note 6, wherein, after the second feature value is calculated, a new second feature value is calculated using a new first feature value corresponding to newly acquired biometric data of the second time unit, and the second feature value having already been calculated.

### (Supplementary Note 8)

The calculation method according to supplementary note 7, wherein, in a case where the second feature value has not been calculated using the first feature value corresponding to biometric data of entire time in the second time unit, every time a subsequent second time unit passes or every time preset time shorter than the second time unit passes, a new second feature value is calculated using a new first feature value corresponding to newly acquired biometric data, and the second feature value having already been calculated.

### (Supplementary Note 9)

A calculating device comprising:
a minimum feature value calculating unit that acquires biometric data obtained by measurement in a time series from a person, and calculates, as a minimum feature value, a feature value of each preset minimum time unit of the acquired biometric data;
a first feature value calculating unit that calculates, after passage of a first time unit which is a time unit longer than the minimum time unit, as a first feature value, a feature value of biometric data obtained by measurement in the first time unit using the minimum feature value corresponding to the biometric data of the first time unit; and
a calculating unit that calculates a value representing physical condition of the person using information based on the first feature value.

### (Supplementary Note 10)

The calculating device according to supplementary note 9, wherein, every time biometric data is acquired, the minimum feature value calculating unit calculates a feature value of each minimum time unit of the acquired biometric data as the minimum feature value.

### (Supplementary Note 11)

The calculating device according to supplementary note 9 or 10, wherein, after the first feature value is calculated, the first feature value calculating unit calculates a new first feature value using a new minimum feature value corresponding to newly acquired biometric data of the first time unit, and the first feature value having already been calculated, and stores the new first feature value.

### (Supplementary Note 12)

The calculating device according to supplementary note 11, wherein, in a case where the first feature value has not been calculated using the minimum feature value corresponding to biometric data of entire time in the first time unit, the first feature value calculating unit checks whether or not biometric data of the first time unit has been newly acquired, and, in a case where biometric data has been newly acquired, the first feature value calculating unit calculates a new first feature value using a new minimum feature value corresponding to the newly acquired biometric data, and the first feature value having already been calculated.

### (Supplementary Note 13)

The calculating device according to supplementary note 12, wherein, in a case where the first feature value has not been calculated using the minimum feature value corresponding to biometric data of entire time in the first time unit, the first feature value calculating unit checks whether or not biometric data of the first time unit has been newly acquired at a time interval shorter than the first time unit, and, in a case where biometric data has been newly acquired, the first feature value calculating unit calculates a new first feature value using a new minimum feature value corresponding to the newly acquired biometric data, and the first feature value having already been calculated.

### (Supplementary Note 14)

The calculating device according to any one of supplementary notes 9 to 13, wherein
the calculating device comprises a second feature value calculating unit that calculates, after passage of a second time unit which is a time unit longer than the first time unit, as a second feature value, a feature value of biometric data obtained by measurement in the second time unit using the first feature value corresponding to the biometric data included in the second time unit, and
the calculating unit calculates a value representing physical condition on a basis of the second feature value.

### (Supplementary Note 15)

The calculating device according to supplementary note 14, wherein, after the second feature value is calculated, the second feature value calculating unit calculates a new second feature value using a new first feature value corresponding to newly acquired biometric data of the second time unit, and the second feature value having already been calculated.

### (Supplementary Note 16)

The calculating device according to supplementary note 15, wherein, in a case where the second feature value has not been calculated using the first feature value corresponding to biometric data of entire time in the second time unit, every time a subsequent second time unit passes or every time preset time shorter than the second time unit passes, the second feature value calculating unit calculates a new second feature value using a new first feature value corresponding to newly acquired biometric data, and the second feature value having already been calculated.

### (Supplementary Note 17)

A computer readable storage medium having stored thereon a program for causing an information processing device to execute processes of:
acquiring biometric data obtained by measurement in a time series from a person, and calculating, as a minimum feature value, a feature value of each preset minimum time unit of the acquired biometric data;
after passage of a first time unit which is a time unit longer than the minimum time unit, calculating, as a first feature value, a feature value of biometric data obtained by measurement in the first time unit using the minimum feature value corresponding to the biometric data of the first time unit; and
calculating a value representing physical condition of the person using information based on the first feature value.

### REFERENCE SIGNS LIST

10 stress value calculating device
11 data acquiring unit
12 short-time feature value calculating unit
13 feature value calculating unit
14 stress value calculating unit
15 output unit
16 acquired data storage unit
17 feature value storage unit
20 user terminal
30 display device
U person
W wearable terminal
100 calculating device
101 CPU
102 ROM
103 RAM
104 program group
105 storage device
106 drive
107 communication interface
108 input/output interface
109 bus
110 storage medium
111 communication network
121 minimum feature value calculating unit
122 first feature value calculating unit
123 calculating unit

What is claimed is:

1. A calculation method comprising:
acquiring biometric data obtained by measurement in a time series from a person, and calculating, as a minimum feature value, a feature value of each preset minimum time unit of the acquired biometric data;
after passage of a first time unit which is a time unit longer than the minimum time unit, calculating, as a first feature value, a feature value of biometric data obtained by measurement in the first time unit using the minimum feature value corresponding to the biometric data of the first time unit; and
calculating a value representing physical condition of the person using information based on the first feature value.

2. The calculation method according to claim 1, wherein, every time biometric data is acquired, a feature value of each minimum time unit of the acquired biometric data is calculated as the minimum feature value.

3. The calculation method according to claim 1, wherein, after the first feature value is calculated, a new first feature value is calculated using a new minimum feature value corresponding to newly acquired biometric data of the first time unit, and the first feature value having already been calculated.

4. The calculation method according to claim 3, wherein, in a case where the first feature value has not been calculated using the minimum feature value corresponding to biometric data of entire time in the first time unit, it is checked whether or not biometric data of the first time unit has been newly acquired, and, in a case where biometric data has been newly acquired, a new first feature value is calculated using a new minimum feature value corresponding to the newly acquired biometric data, and the first feature value having already been calculated.

5. The calculation method according to claim 4, wherein, in a case where the first feature value has not been calculated using the minimum feature value corresponding to biometric data of entire time in the first time unit, it is checked whether or not biometric data of the first time unit has been newly acquired at a time interval shorter than the first time unit, and, in a case where biometric data has been newly acquired, a new first feature value is calculated using a new minimum feature value corresponding to the newly acquired biometric data, and the first feature value having already been calculated.

6. The calculation method according to claim 1, wherein
the calculation method comprises, after passage of a second time unit which is a time unit longer than the first time unit, calculating, as a second feature value, a feature value of biometric data obtained by measurement in the second time unit using the first feature value corresponding to the biometric data included in the second time unit, and
a value representing physical condition is calculated on a basis of the second feature value.

7. The calculation method according to claim 6, wherein, after the second feature value is calculated, a new second feature value is calculated using a new first feature value corresponding to newly acquired biometric data of the second time unit, and the second feature value having already been calculated.

8. The calculation method according to claim 7, wherein, in a case where the second feature value has not been calculated using the first feature value corresponding to biometric data of entire time in the second time unit, every time a subsequent second time unit passes or every time preset time shorter than the second time unit passes, a new second feature value is calculated using a new first feature value corresponding to newly acquired biometric data, and the second feature value having already been calculated.

9. A calculating device comprising:
at least one memory configured to store instructions; and
at least one processor configured to execute instructions to:
acquire biometric data obtained by measurement in a time series from a person, and calculates, as a minimum feature value, a feature value of each preset minimum time unit of the acquired biometric data;
calculate, after passage of a first time unit which is a time unit longer than the minimum time unit, as a first feature value, a feature value of biometric data obtained by measurement in the first time unit using the minimum feature value corresponding to the biometric data of the first time unit; and
calculate a value representing physical condition of the person using information based on the first feature value.

10. The calculating device according to claim 9, wherein, the at least one processor is configured to execute the instructions to, every time biometric data is acquired, calculate a feature value of each minimum time unit of the acquired biometric data as the minimum feature value.

11. The calculating device according to claim 9, wherein the at least one processor is configured to execute the instructions to, after the first feature value is calculated, calculate a new first feature value using a new minimum feature value corresponding to newly acquired biometric data of the first time unit, and the first feature value having already been calculated.

12. The calculating device according to claim 11, wherein the at least one processor is configured to execute the instructions to, in a case where the first feature value has not been calculated using the minimum feature value corresponding to biometric data of entire time in the first time unit, check whether or not biometric data of the first time unit has been newly acquired, and, in a case where biometric data has been newly acquired, calculate a new first feature value using a new minimum feature value corresponding to the newly acquired biometric data, and the first feature value having already been calculated.

13. The calculating device according to claim 12, wherein the at least one processor is configured to execute the instructions to, in a case where the first feature value has not been calculated using the minimum feature value corresponding to biometric data of entire time in the first time unit, check whether or not biometric data of the first time unit has been newly acquired at a time interval shorter than the first time unit, and, in a case where biometric data has been newly acquired, calculate a new first feature value using a new minimum feature value corresponding to the newly acquired biometric data, and the first feature value having already been calculated.

14. The calculating device according to claim 9, wherein
the at least one processor is configured to execute the instructions to:
calculate, after passage of a second time unit which is a time unit longer than the first time unit, as a second feature value, a feature value of biometric data obtained by measurement in the second time unit using the first feature value corresponding to the biometric data included in the second time unit, and
calculate a value representing physical condition on a basis of the second feature value.

15. The calculating device according to claim 14, wherein the at least one processor is configured to execute the instructions to, after the second feature value is calculated, calculate a new second feature value using a new first feature value corresponding to newly acquired biometric data of the second time unit, and the second feature value having already been calculated.

16. The calculating device according to claim 15, wherein the at least one processor is configured to execute the instructions to, in a case where the second feature value has not been calculated using the first feature value corresponding to biometric data of entire time in the second time unit, every time a subsequent second time unit passes or every time preset time shorter than the second time unit passes, calculate a new second feature value using a new first feature value corresponding to newly acquired biometric data, and the second feature value having already been calculated.

17. A non-transitory computer readable storage medium having stored thereon a program comprising instructions for causing an information processing device to execute processes of:
acquiring biometric data obtained by measurement in a time series from a person, and calculating, as a minimum feature value, a feature value of each preset minimum time unit of the acquired biometric data;
after passage of a first time unit which is a time unit longer than the minimum time unit, calculating, as a first feature value, a feature value of biometric data obtained by measurement in the first time unit using the minimum feature value corresponding to the biometric data of the first time unit; and
calculating a value representing physical condition of the person using information based on the first feature value.
